(19) United States
(12) Patent Application Publication
(10) Pub. No.: US 2025/0312416 A1
(43) Pub. Date: Oct. 9, 2025

(54) METHOD AND PHARMACEUTICAL COMPOSITION FOR CARTILAGE REGENERATION

(71) Applicant: FAR EASTERN MEMORIAL HOSPITAL, New Taipei City (TW)

(72) Inventors: Hsiu-Jung LIAO, New Taipei City (TW); Chih-Hung CHANG, New Taipei City (TW); Yi-Shan SHEN, New Taipei City (TW); Chi-Ying HUANG, New Taipei City (TW)

(73) Assignee: FAR EASTERN MEMORIAL HOSPITAL, New Taipei City (TW)

(21) Appl. No.: 19/098,886

(22) Filed: Apr. 2, 2025

Related U.S. Application Data
(60) Provisional application No. 63/575,293, filed on Apr. 5, 2024.

(51) Int. Cl. A61K 38/19; A61K 35/28; A61P 19/02; A61P 19/04; C12Q 1/6883

(52) U.S. Cl. CPC A61K 38/195; A61K 35/28; A61P 19/02; A61P 19/04; C12Q 1/6883; C12Q 2600/158

(57) ABSTRACT

The present invention is related to a method for treating a cartilage defect disease in a subject comprising administering said subject with using a CXCR4 agonist. Also provided is the pharmaceutical composition for treating a cartilage defect disease comprising a CXCR4 agonist.

### CROSS REFERENCE TO RELATED APPLICATION

This application claims priority to U.S. Provisional Application No. 63/575,293, filed on Apr. 5, 2024, the content of which is hereby incorporated by reference in its entirety.

### REFERENCE TO ELECTRONIC SEQUENCE LISTING

The application contains a Sequence Listing which has been submitted electronically in .XML format and is hereby incorporated by reference in its entirety. Said .XML copy, created on Apr. 2, 2025, is named “FEH0007US-Sequence Listing.xml” and is 8,128 bytes in size. The sequence listing contained in this .XML file is part of the specification and is hereby incorporated by reference herein in its entirety.

### FIELD OF THE INVENTION

The invention concerns a new method and pharmaceutical composition for cartilage regeneration.

### BACKGROUND OF THE INVENTION

Cartilage is a tissue composed of extracellular matrix and cartilage cells as chondrocyte, which has neither no blood vessels nor nerve. Therefore, it is difficult to heal the cartilage tissue when it is damaged. Further, chondrocytes are surrounded by hard extracellular matrix, and thus it is difficult to regenerate cartilage once damaged or degenerated.

The interactions of varieties of growth factors, cytokines, and signaling molecules have been reported to regulate the chondrogenic differentiation of mesenchymal stromal cells [1]. Transforming growth factor-beta (TGF-β) superfamily members is crucial for the in vitro chondrogenic commitment of mesenchymal stromal cells. However, the upregulation of TGF-β contributes to cell degeneration and inflammation that had been reported to interfere with chondrogenesis and has a risk of inducing fibrotic side effects [2-4]. On the other hand, accumulative findings from analysis of the expression of Wnt signaling molecules and in vivo and in vitro functional experiments suggest that inhibition of Wnt signaling is a therapeutic target for OA [5, 6]. However, the WNT inhibitors have been reported in the early clinical stages as an increase in bone remodeling with off-target side effects. It may imply that aberrant Wnt signaling would cause or be closely associated with skeletal disorders such as dwarfism, deformity of skeletons, osteoporosis, and high bone-mass syndrome [7].

Accordingly, it is desirable to develop a state-of-the-art molecule as a new therapeutic target that regulates chondrogenesis and cartilage repair in treating osteoarthritic articular cartilages.

### SUMMARY OF THE INVENTION

Accordingly, the present invention provides a new approach to enhance the cartilage regeneration by using a protein or peptide, and thus which can be developed as a new drug for treating cartilage damages.

In one aspect, the present invention provides a method for cartilage regeneration in a subject, which comprises administering said subject a therapeutically effective amount of a composition comprising a CXCR4 agonist, wherein the agonist has a CXCR4 agonistic activity that induces chondrogenesis.

In one embodiment of the invention, the agonist is CXCL14 protein, or a variant, variable domain, functional derivative, or fragment thereof, which has the same function of CXCL14, a genetically modified recombinant protein or chemically modified protein thereof, or a gene construct for expressions of CXCL14.

In another embodiment of the invention, the agonist is CXCL12 protein, or a variant, variable domain, functional derivative, or fragment thereof, which has the same function of CXCL12, a genetically modified recombinant protein or chemically modified protein thereof, or a gene construct for expressions of CXCL12.

In one further aspect, the present invention provides a method for treating a cartilage defect disease in a subject, comprising administering said subject a therapeutically effective amount of a composition comprising a CXCR4 agonist, wherein the agonist has a CXCR4 agonistic activity that induces chondrogenesis.

In one embodiment of the invention, the cartilage defect disease is degenerative osteoarthritis.

In a yet aspect, the present invention provides a method for treating a cartilage defect disease in a subject, comprising administering said subject with a cell preparation obtained by culturing cells or stem cells treated with a CXCR4 agonist, wherein the agonist has a CXCR4 agonistic activity that induces chondrogenesis.

In a further aspect, the present invention provides a method for treating a cartilage defect disease in a subject, comprising administering said subject with a cell medium treated with a CXCR4 agonist, wherein the agonist has a CXCR4 agonistic activity that induce chondrogenesis.

In the present invention, the cell medium contains the secretome derived from the cells treated with a CXCR4 agonist.

In one yet aspect, the invention provides a method for treating a cartilage defect disease in a subject, comprising administering said subject with a cell preparation containing cells treated with extracellular vesicles (EVs) released from the cells treated with a CXCR4 agonist, wherein the agonist has a CXCR4 agonistic activity.

In one embodiment of the invention, the cells are stem cells, including induced pluripotent stem cells.

In one example of the invention, the stem cells are mesenchymal stem cells (MSCs), including but not limited to Wharton's jelly-derived MSCs (WJ-MSCs), infrapatellar fat pad-derived MSCs (IPFP-MSCs), subcutaneous adipose tissue-derived MSCs (SC-MSCs), amniotic fluid-derived MSCs (AF-MSCs), bone marrow-derived MSCs (BM-MSCs), umbilical cord-derived MSCs (UC-MSCs).

In the invention, the method for treating a cartilage defect disease in a subject, comprises a combination of two or more of the above-mentioned methods.

In one further yet aspect, the present invention provides a method for detecting a cartilage defect disease, wherein the gene expression signatures of CXCL14, CXCL12 or CXCR4 is used as a biomarker for treating a cartilage defect disease.

In one example of the invention, the CXCL14 protein (CXCL14-P) has the amino acid sequence of ITTKSVSRYRGQEH (SEQ ID NO:1).

In one example of the invention, the CXCL12 protein (CXCL12-P) has the amino acid sequence of RANVKHLKILN (SEQ ID NO:2).

In one example of the invention, the CXCR4 protein (CXCR4-P) has the amino acid sequence of MGYQKKLRSMTDKYRL (SEQ ID NO:3).

It is to be understood that both the foregoing general description and the following detailed description are exemplary and explanatory only and are not restrictive of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing description and the following detailed description of the invention will be better understood when reading in conjunction with the accompanying drawings. For the purpose of illustrating the present invention, currently preferred embodiments are shown in the drawings.

FIGS. 1A-1E shows the efficacy of CXCL14 in stimulating cartilage repair, providing the selected genes of microarray data at day 28 from human bone marrow cells during chondrogenesis. The GSE140861 microarray database was used for analyzing the potential genes from human bone marrow cells at different time points during chondrogenesis. Whole-genome expression analysis was carried out using Illumina (CA) Human Ref-8v3 or Human HT-12 v4 BeadArrays™. It was first selected the secreted protein from this database and further verified the functions of the candidate genes. The numbers of significant differences in gene expressions of different time points from human bone marrow stem cells during chondrogenesis. FIG. 1A provides the data visualized using a volcano plot with red dots indicating statistically significantly different proteins with an abundance change of 2-fold or higher. FIG. 1B shows that CXCL14 increased the chondrogenic potential. Alcian blue staining analyzed the chondrogenic differentiation of pellets with varying ratios of IPFP-MSC. CXCL14 increased the GAG deposition in IPFP-MSC during chondrogenesis on day 3. FIG. 1C provides the fluorescence confocal microscopy images showing IPFP-MSC spheroids from OA patients treated with CXCL14 for 7 days; wherein the nuclei were stained with DAPI (blue), and merged images display SOX9 (red), type II collagen (green), and aggrecan (light blue) labeling. Both side views (observed in the x and z plane) and bottom views (observed in the y and z plane) of the images are presented. Scale bar=500 μm. FIG. 1D provides the Safranin O staining and IHC staining of collagen II in the tibial articular cartilage of WT mice after ACLT (Scale bars, 100 μm); wherein the Safranin O staining and IHC staining of collagen II in mouse joints (n=6) with sham, OA cartilage, and receiving CXCL14. Black arrows indicate reduced cartilage thickness and staining intensity in OA mice. F, femur; T, tibia. OARSI scores indicated articular cartilage damage/repair in all groups. The OARSI score of the ACLT group (score of 5.5) was significantly higher than that of the sham group (score of 0.17). CXCL14 administration can improve the score to 2.7 (10 ng) and 1 (20 ng). FIG. 1E shows that the combination of CXCL14 and TGF-b increased the chondrogenic potential. The chondrogenic progenitor cells in the human IPFP-MSC from OA patients were detected using SOX9 and Collagen type II biomarkers. MSCs were assessed by flow cytometry for SOX9 and Collagen type II biomarkers indicative of human MSCs on day 1. In addition, Alcian blue staining analyzed the chondrogenic differentiation of pellets with IPFP-MSC. CXCL14 and TGF-b-treated IPFP-MSCs had higher GAG contents consistent with the higher SOX9+ type II collagen+-expressing population during chondrogenesis.

FIGS. 2A, 2B and 2C show the identification of functional peptide sequences within the CXCL14 protein for enhancing chondrogenesis by utilizing AlphaFold2-Multimer prediction software. FIG. 2A shows that the structural alignment was shown between the CXCL14 and CXCR4 regions of the AlphaFold 2 model (highlighted in orange). FIG. 2B shows that the Predicted Alignment Error (PAE) was illustrated for the top-ranked model. The color at coordinates (x, y) represented the anticipated distance error in the position of residue x when aligning the predicted structure with the true structure on residue y. FIG. 2C shows that the predicted amino acid sequencing was provided for the region between CXCL14 and CXCR4.

FIGS. 3A and 3B show the predicted peptide sequences for CXCL14 and CXCL12. FIG. 3A provides the predicted peptide sequences of CXCL14, and FIG. 3B provides the predicted peptide sequences of CXCL12, which were displayed and synthesized to validate the chondrogenic potential of MSCs.

FIG. 4 shows the peptides derived from CXCL12/CXCL14 had a higher capacity to induce chondrogenesis when compared to the CXCL12/CXCL14 proteins. In clinical-grade ADSCs, CXCL12/CXCL14 peptides significantly upregulated the expression of chondrogenic genes compared to CXCL12/CXCL14 proteins. Real-time PCR analysis was conducted on day 7 for CXCL12/CXCL14 peptides treated ADSCs, evaluating the expression of COMP, SOX9, COL2A1, ACAN, ITGB1, and SOSTDC1 genes. Cells were harvested, and RNA extraction was performed using a LightCycler® (Roche Applied Science). The fold change in gene expression on the y-axis was calculated relative to the same gene expression in untreated chondrocytes. Statistical analysis was conducted using one-way ANOVA. Significance levels are denoted as follows: * p<0.05, ** p<0.01, *** p<0.001, and **** p<0.0001 for comparisons between PBS treatments and the untreated group.

FIG. 5 shows the functional peptides derived from CXCL14/CXCL12 exhibited the increased GAG content induction. The chondrogenic differentiation of pellets containing clinical-grade ADSCs was assessed through Alcian blue staining. Clinical-grade ADSCs treated with CXCL14/CXCL12 peptides demonstrated significantly higher GAG content than those treated with CXCL14/CXCL12 proteins during the process of chondrogenesis.

FIG. 6 shows CXCL14/CXCL12 stimulated SOX9, type II collagen, and aggrecan levels in ADSC spheroids by fluorescence confocal microscopy. CXCL14/CXCL12 proteins notably induced the expression of SOX9, type II collagen, and aggrecan in ADSC for 7 days. Nuclei were stained with DAPI (blue), and merged images exhibit the labeling of SOX9 (red), type II collagen (green), and aggrecan (light blue). Both side views (observed in the x and z plane) and bottom views (observed in the y and z plane) of the images are provided. The scale bar is set at 500 μm.

FIG. 7 shows that the allosteric agonist of CXCR4 induces chondrogenesis. The CXCR4 agonist showed a superior capacity to induce chondrogenesis. In clinical-grade ADSCs, CXCR4 agonists significantly elevated the expression of chondrogenic genes. Real-time PCR analysis was performed on day 7 for ADSCs treated with CXCR4 agonists, assessing the expression of COMP, SOX9, COL2A1, ACAN, ITGB1, CXCL14, and SOSTIC1 genes. Cellular harvest, RNA extraction, and gene expression quantification were conducted using a LightCycler® (Roche Applied Science). The fold change in gene expression on the y-axis was calculated relative to the same gene expression in untreated chondrocytes. Statistical analysis was executed through one-way ANOVA. Significance levels are represented as follows: * p<0.05, ** p<0.01, *** p<0.001, and **** p<0.0001, indicating the significance of comparisons between PBS treatments and the untreated group.

FIG. 8 shows that the allosteric agonist of CXCR4 induced GAG content during chondrogenesis. The chondrogenic differentiation of pellets containing clinical-grade ADSCs was evaluated using Alcian blue staining. Remarkably, clinical-grade ADSCs treated with CXCR4 agonists exhibited notably higher GAG content than those treated without CXCR4 agonists during the chondrogenesis process.

FIGS. 9A-9C show that the ADSC spheroids induced by CXCL12/CXCL14 can promote cartilage regeneration. FIG. 9A shows that the knee swelling was reduced following intraarticular administration of CXCL12/CXCL14-ADSC spheroids. The electrical shock delivered by the grid in the treadmill was of constant intensity. The number of electric shocks during treadmill exercise was significantly reduced by intraarticular administration of CXCL12/CXCL14-ADSC spheroids compared to the OA group treated with PBS. FIG. 9B provides the Safranin O staining of the tibial articular cartilage in rats after ACLT (Scale bars, 100 μm). Safranin O staining was performed in rat joints (n=6) from the sham group, OA cartilage, and those receiving CXCL12/CXCL14-ADSC spheroids. FIG. 9C shows the OARSI scores, which indicate the degree of articular cartilage damage and repair, were assessed for all groups. The ACLT group showed a significantly higher OARSI score (5.61) compared to the sham group (0.17). Intraarticular administration of CXCL12/CXCL14-ADSC spheroids (1.5×10⁶ cells) improved the OARSI scores, reducing them to 2.06 and 2.56, respectively.

FIGS. 10A-10C show the temporal progression of chondrogenic induction in ADSCs following treatment with CXCL14 peptide (CXCL14-P)/CXCL12 peptide (CXCL12-P). FIG. 10A provides the flowchart of the protocol for peptide treatment and sample collection. FIG. 10B shows that CXCL14-P and CXCL12-P demonstrated superior ability to promote chondrogenesis. TGF-beta affects the expression of chondrogenic genes, including ITGB1 and COMP. In clinical-grade ADSCs, these peptides significantly upregulated the expression of chondrogenic genes. On day 7 of treatment with CXCL14-P or CXCL12-P, real-time PCR analysis revealed increased expression of COMP, SOX9, COL2A1, ACAN, and ITGB1. FIG. 10C shows that Alcian blue staining was used to evaluate the chondrogenic differentiation of pellets containing clinical-grade ADSCs. Treatment with CXCL14/CXCL12 peptides resulted in significantly increased GAG content during chondrogenesis in the presence of TGF-beta.

FIG. 11 shows the peptides labeled with fluorescent markers penetrated ADSC. Cell-penetrating properties of CXCL14-P and CXCL12-P peptides in ADSCs were analyzed. ADSCs were incubated with 10 ng of FITC-conjugated peptides at 37° C. for one day, followed by refreshing the chondrogenic medium. Peptide internalization was evaluated through fluorescence microscopy on days 1, 3, and 7.

FIG. 12 shows that TGF-beta can significantly enhance chondrogenic phenotypes in ADSCs but not UC-MSCs. In clinical-grade ADSCs and UC-MSCs, TGF-beta significantly upregulated chondrogenic gene expression. By day 7 of TGF-beta treatment, real-time PCR analysis showed increased expression of COMP, SOX9, COL2A1, ACAN, and ITGB1 in ADSCs.

FIGS. 13A, 13B and 13C show that CXCL14-P and CXCL12-P can still enhance chondrogenic differentiation even under low TGF-beta responsiveness in UC-MSCs. FIG. 13A provides flowchart illustrating the protocol for peptide treatment and sample collection. FIG. 13B shows that CXCL14-P and CXCL12-P exhibited exceptional capacity to enhance chondrogenesis. TGF-beta influenced the expression of chondrogenic genes, including ITGB1 and COMP. In UC-MSCs, these peptides significantly upregulated chondrogenic gene expression. On day 7 of treatment with CXCL14-P or CXCL12-P, real-time PCR analysis revealed elevated levels of COMP, SOX9, COL2A1, ACAN, and ITGB1. FIG. 13C shows that Alcian blue staining was performed to assess the chondrogenic differentiation of pellets containing UC-MSCs. Treatment with CXCL14/CXCL12 peptides increased GAG content during chondrogenesis in the presence of TGF-beta.

FIGS. 14A and 14B shows that CXCL14 peptide-treated ADSC-derived EVs significantly enhance chondrogenesis. FIG. 14A shows that the expression levels of Collagen Type II (green), SOX9 (red), COMP (indigo), and Hoechst 33342 (gray) in 3D ADSC spheroids treated with MSC-EV-CXCL14-P at various time points were assessed using immunofluorescence staining. FIG. 14B shows that the knee swelling was reduced following intra-articular administration of MSC-EVs, MSC-EVs containing SOX9, or CXCL14-P-MSC-EVs. The treadmill grid's electrical shock intensity remained constant, but the number of shocks received during treadmill exercise was significantly lower in the groups treated with MSC-EVs, MSC-EV-SOX9, or CXCL14-P-MSC-EVs compared to the OA group treated with PBS.

FIGS. 15A and 15B show the characterization of CXCL14 peptides. FIG. 15A shows that the cellular uptake of FAM-labeled CXCL14-P in ADSCs was visualized using fluorescence microscopy. ADSCs were treated with 10 ng of FAM-CXCL14-P (green) and incubated at 37° C. for 24 hours. The culture medium was then replaced with fresh medium without FAM-CXCL14-P, and the cells were monitored up to day 7. FIG. 15B shows that a peptide binding assay was conducted by treating ADSCs with 1, 10, or 100 ng of CXCL14-biotin. The binding was analyzed in a time-dependent manner using an ELISA reader.

FIGS. 16A, 16B and 16C show that the alanine substitution with positively or negatively charged amino acids in CXCR4 and CXCL14 peptides can further enhance their ability to stimulate chondrogenesis. FIG. 16A shows that the positively or negatively charged alanine-substituted variants of CXCL12, CXCL14, and CXCR4 peptides, including CXCL12-PM1, CXCL14-PM1, CXCL14-PM2, CXCL14-PM3, and CXCR4-PM1, were synthesized and found to significantly enhance the expression of chondrogenic induction. FIG. 16B shows that CXCL14-PM1, CXCL14-PM2, CXCL14-PM3 (v.s. CXCL14-P), and CXCR4-PM1 (v.s. control) enhanced chondrogenic potential. Alcian blue staining was used to assess the chondrogenic differentiation of ADSC pellets. On day 7, these variants increased GAG deposition in ADSCs during chondrogenesis. FIG. 16C shows that chondrogenic induction in ADSCs was achieved following treatment with these alanine-substituted peptide modifications. Treatment with CXCL14-PM1, CXCL14-PM2, CXCL14-PM3, and CXCR4-PM1 was sufficient to enhance the expression of chondrogenic genes in ADSCs. On day 7, real-time PCR was performed to analyze the expression of key chondrogenic markers, including SOX9, COL2A1, COMP, ACAN, ITGB1, CXCL14, and CXCR4, in ADSCs treated with these modified peptides. ADSCs were exposed to distinct peptide modifications derived from CXCL12, CXCL14, and CXCR4 at a concentration of 10 ng for a one-day treatment, after which the medium was replaced with fresh peptide-free medium. At the end of the treatment period, RNA was extracted to evaluate the expression levels of chondrogenesis-related cytokine genes. Gene expression quantification was performed using a LightCycler system (Roche Applied Science), and fold changes were calculated relative to untreated control ADSCs. The results shown on the Y-axis demonstrate the modulatory effects of CXCL14-PM1, CXCL14-PM2, CXCL14-PM3, and CXCR4-PM1 on chondrogenic gene expression in ADSCs, offering insights into their potential roles in promoting chondrogenesis. FIG. 16(D) provides a summary of the results for alanine substitution with positively or negatively charged amino acids in CXCL12, CXCL14, and CXCR4 peptides.

FIG. 17A-17C shows that modification of CXCL14-P enhances stability and chondrogenic potential. FIG. 17A shows that modified CXCL14 peptides, including AC-CXCL14-P-NH2 (referred to as CXCL14-P1) and H-CXCL14-P-OH (referred to as CXCL14-P2), were synthesized and not found to significantly upregulate chondrogenic gene expression. ADSCs were treated with CXCL14-P, CXCL14-P1, or CXCL14-P2, respectively, at a concentration of 10 ng for 24 hours, after which the medium was replaced with fresh peptide-free medium. On day 7, total RNA was extracted, and real-time PCR analysis was performed to assess the expression levels of key chondrogenic markers, including ACAN, COL2A1, SOX9, and COMP. Gene expression levels were quantified using a LightCycler system (Roche Applied Science), with fold changes calculated relative to untreated control ADSCs. FIG. 17B shows that the spheroid formation during chondrogenesis in response to peptide treatments did not induce cell death, as ADSCs in spheroids were stained with calcein-AM (live) and P1 (dead) dyes to assess cell viability (top panel). Immunofluorescence staining was performed to detect Collagen Type II, SOX9, COMP, and nuclei (Hoechst 33342) in 3D ADSC spheroids (middle panel). Alcian blue staining revealed that CXCL14-P1 and CXCL14-P2 significantly increased GAG deposition in ADSCs on day 7 compared to CXCL14-P, indicating enhanced chondrogenic differentiation (bottom panel). FIG. 17C shows that CXCL14-P1 and CXCL14-P2 significantly improved the chondrogenic potential of ADSCs. Alcian blue staining confirmed greater GAG accumulation in ADSC pellets treated with CXCL14-P1 and CXCL14-P2 on day 7, demonstrating superior chondrogenic differentiation compared to CXCL14-P.

### DETAILED DESCRIPTION

Unless otherwise defined, all technical and scientific terms used herein have the same meaning as commonly understood by those skilled in the art to which the present invention pertains. It should be understood that the terminology used herein is for the purpose of describing specific embodiments only and is not intended to be limiting.

As used herein, the singular forms “a”, “an” and “the” include plural references unless explicitly indicated otherwise. Thus, for example, reference to “a sample” includes a plurality of such samples and their equivalents known to those skilled in the art.

As used herein, the term “cartilage” includes hyaline cartilage, fibrocartilage, or elastic cartilage, and is not particularly limited. One particular example in the present invention, is knee cartilage.

As used herein, the term “cartilage damage” refers to a disease caused by cartilage defects, injuries, damages, or damages caused by cartilage, cartilage tissue and/or joint tissues (synovial membrane, articular capsule, cartilaginous bone, etc.) injured by mechanical stimulation or inflammatory reaction. Such cartilage defect diseases include, but are not limited to, degenerative arthritis, rheumatoid arthritis, fractures, muscle tissue damage, plantar fasciitis, humerus ulcer, calcified myositis, or joint damage caused by fracture nonunion or trauma. In the invention, one particular example is osteoarthritis (OA).

As used herein, the phrase “therapeutically effective amount” refers to an amount sufficient to treat a disease at a reasonable benefit/risk ratio applicable to medical treatment, and an effective dose level may be determined by the species and severity, age, sex, type of disease, duration of treatment, factors including co-administered drugs, and factors well known in other medical disciplines.

As used herein, the term “subject” refers to a human or any animal, including a human or an animal particularly pet animals such as dogs, cats, horses and etc., with a cartilage damage, and indicates a group in need of a treatment for improving cartilage regeneration or a treatment of a cartilage damage.

As used herein, the term “Chemokine (C—X—C motif) ligand 14”, “CXCL14 protein” or “CXCL14” refers to a small cytokine belonging to the CXC chemokine family that is also known as BRAK (for breast and kidney-expressed chemokine), which mainly regulates immune cell migration and executes antimicrobial immunity. Yet, the underlying chondrogenic functions and mechanisms are still unknown. In the present invention, one example of CXCL14 used in the present invention is Homo sapiens CXCL14. One example of CXCL14 protein (CXCL14-P) is the protein having the amino acid sequence of ITTKSVSRYRGQEH as set forth in SEQ ID NO:1. The CXCL14 proteins also provide the variants thereof, such as CXCL14-PM1 having the amino acid sequence of ITTASVSAYAGQEH as set forth in SEQ ID NO: 4; CXCL14-PM2 having the amino acid sequence of ITTKSVSRYRGQAH as set forth in SEQ ID NO: 5; and CXCL14-PM3 having the amino acid sequence of ITTKSVSRYRGDEH as set forth in SEQ ID NO: 6.

As used herein, the term “CXCL14 gene” or “CXCL14” means the gene coding for the CXCL14 protein, or the CXCL14 mRNA, such as the Homo sapiens CXCL14 mRNA referring to NCBI Reference Sequence: NM_004887.5.

As used herein, the term “C—X—C motif chemokine 12”, “CXCL12 protein” or “CXCL12,” as known as stromal cell-derived factor 1 (SDF-1), refers to a chemokine protein that in humans is encoded by the CXCL12 gene on chromosome 10. The CXCL12 proteins belongs to the group of CXC chemokines, whose initial pair of cysteines are separated by one intervening amino acid. One example of CXCL12 protein (CXCL12-P) is the protein having the amino acid sequence of RANVKHLKILN as set forth in SEQ ID NO: 2. The CXCL12 proteins also provide the variants thereof, such as CXCL12-PM1 having the amino acid sequence of AANVAHLAILN as set forth in SEQ ID NO: 7.

As used herein, the term “CXCR4” refers to a protein C—X-C chemokine receptor type 4 (CXCR-4) also known as fusion or CD184 (cluster of differentiation 184), which is a protein in humans encoded by the CXCR4 gene. CXCR4 is present in newly generated neurons during embryogenesis and adult life where it plays a role in neuronal guidance. The levels of the receptor decrease as neurons mature. CXCR4 mutant mice have aberrant neuronal distribution. This has been implicated in disorders such as epilepsy. One example of CXCR4 protein (CXCR4-P) is the protein having the amino acid sequence of MGYGKKLRSMTDKYRL as set forth in SEQ ID NO: 3. The CXCR4 protein also provide the variants thereof, such as CXCR4-PM1 having the amino acid sequence of MGYGAALASMTDAYAL as set forth in SEQ ID NO: 8.

As used herein, the term “CXCR4 agonist” refers to a compound which binds to and activates the CXCR4, including for example, CXCL12 or CXCL14, or a variant, variable domain, functional derivative, or fragment thereof, or a genetically modified recombinant protein or chemically modified protein thereof, which has the same function of CXCL12 or CXCL14.

In one example of the present invention, the CXCR4 agonist is a peptide compound, i.e., a peptide or protein.

As used herein, the term “CXCR4 agonistic activity” refers to the activation of the CXCR4.

In one example of the present invention, the CXCR4 agonist is CXCL14 protein, or a variant, variable domain, functional derivative, or fragment thereof having the same function of CXCL14, a genetically modified recombinant protein or chemically modified protein thereof, or a gene construct for expressions of CXCL14.

In one example of the present invention, the CXCR4 agonist is a CXCL12 protein, or a variant, variable domain, functional derivative, or fragment thereof having the same function of CXCL12, a genetically modified recombinant protein or chemically modified protein thereof, or a gene construct for expressions of CXCL12.

As used herein, the term “extracellular vesicles” or “EVs” refers to lipid bilayer-delimited particles that are naturally released from almost all types of cells but, unlike a cell, cannot replicate. EVs can be divided according to size and synthesis route into exosomes, microvesicles and apoptotic bodies. The composition of EVs varies depending on their parent cells, encompassing proteins (e.g., adhesion molecules, cytoskeletons, cytokines, ribosomal proteins, growth factors, and metabolic enzymes), lipids (including cholesterol, lipid rafts, and ceramides), nucleic acids (such as DNA, mRNA, and miRNA), metabolites, and even organelles. A wide variety of EV subtypes have been proposed, defined variously by size, biogenesis pathway, cargo, cellular source, and function, leading to a historically heterogenous nomenclature including terms like exosomes and ectosomes.

In the present invention, the chondrogenic differentiation of hBM-MSC was confirmed and the selecting potential genes regulating the induction of chondrogenesis were verified by exploiting microarray gene expressions. Microarray data showed that the (′XCL14 gene, which highly expressed in hBM-MSC during chondrogenesis. CXCL14, that is a novel chemokine, mainly regulates immune cell migration and executes antimicrobial immunity. Yet, the underlying chondrogenic functions and mechanisms are still unknown. It was ascertained in the invention that CXCL14 could enhance chondrogenesis and would correlate with dynamic changes during chondrogenesis, and the mechanisms.

It was demonstrated in the present invention that the human infrapatellar fat-pad MSC (IPFP-MSC) chondrogenesis can be significantly improved by treating the recombinant protein CXCL14 and cartilage regeneration in an ACLT-induced OA mouse model. Furthermore, highly detected chondrogenic genes co-treated with a low dose of TGF-b showed CXCL14 significantly stimulated chondrogenic induction, suggesting that TGF-b downstream signaling might strengthen the CXCL14 in regulating the chondrogenic capacity. CXCR4 serves as a primary receptor for CXCL14 [11], and CXCL12 can also bind to CXCR4 [12], thereby inducing chondrogenesis. In the invention, the functional peptides derived from CXCL14/CXCL12 were synthesized, which reveal a more pronounced potential to augment chondrogenesis compared to the complete sequences of CXCL14/CXCL12.

The present invention initially unveiled the involvement of CXCL14/CXCL12-activated CXCR4 signaling in regulating chondrogenic induction from MSCs. Furthermore, these potential targets demonstrated efficacy in alleviating OA progression induced by ACLT. Thus, our findings suggest that the CXCL14/CXCL12 peptides can influence chondrogenesis induction and may serve as promising candidates for OA therapy. Moreover, we aim to understand the mechanism of CXCR4 signaling activation in regulating chondrogenesis. The results could provide an understanding of the functions and regulation of chondrocyte differentiation by CXCR4 signaling activation and shed light on the identification of new therapeutic targets.

In one particular example of the present invention, the cartilage damage is osteoarthritis (OA).

In the method of the invention, the composition for cartilage regeneration or treatment of cartilage defect disease comprises:
1. CXCL14 or CXCL12 protein/peptide (CXCL14-P or CXCL12-P);
2. the cells with CXCL14 or CXCL12 protein/peptide;
3. the secretome which is derived from the cells treated with CXCL14 or CXCL12 protein/peptide;
4. The extracellular vesicles (EVs) which are released from the cells treated with CXCL14 or CXCL12 protein/peptide;
5. a variant, variable domain, functional derivative, or fragment thereof which has the same function of CXCL14 or CXCL12, for example, CXCL14-PM1, CXCL14-PM2, CXCL14-PM3, or CXCL12-PM1;
6. a genetically modified recombinant protein or chemically modified protein thereof, which has the same function of CXCL14 or CXCL12,
7. a gene construct for expressions of CXCL14 or CXCL12; or
8. A combination of any two or more of the aforementioned approaches.

According to the invention, the stem cells can be treated with a CXCR4 agonist to prepare a cell preparation for use in the method for cartilage regeneration. Various types of MSCs can be used, including for example, Wharton's jelly-derived MSCs (WJ-MSCs), infrapatellar fat pad-derived MSCs (IPFP-MSCs), subcutaneous adipose tissue-derived MSCs (SC-MSCs), amniotic fluid-derived MSCs (AF-MSCs), bone marrow-derived MSCs (BM-MSCs), and umbilical cord-derived MSCs (UC-MSCs), which have been investigated for its potential in inducing chondrogenesis. It is ascertained in the present invention that CXCL14, CXCL12, or CXCR4 plays a role in regulating cell differentiation, such as osteoclasts and osteoblasts, was also explored for its potency.

### EXAMPLES

### Material and Methods

### 1. Research Ethics and Informed Consent

This study was recruited patients aged 50 to 75 undergoing joint replacement surgery, comprising both non-OA and OA individuals (n=6). IPFPs were promptly obtained and processed. All participants were required to sign informed consent forms to utilize their IPFP tissue. Patients with rheumatic diseases treated with immunosuppressive drugs in the past three months were excluded. Approval for the study was granted by the Research Ethics Committee of Far Eastern Memorial Hospital, Taipci (111225-F), Taiwan.

### 2. Evaluation of IPFP-MSC Differentiation Ability

To assess differentiation capacity, single-cell suspensions from cultured IPFP-MSCs and ADSCs were cultivated in chondrogenic medium. Alcian Blue staining was performed on day 7 to evaluate chondrogenic differentiation. The chondrogenic differentiation test was measured gene expressions of ACAN, COL2A1, SOX9, COMP, ITGB1, CXCL14 and SOSTDC1.

### 3. Quantitative Real-Time PCR for Gene Expression Profiles of Inflammation and Chondrogenesis

Human IPFP-MSCs and ADSCs were expanded in Dulbecco's Modified Eagle's Medium (DMEM, Gibco, cat. no 2145484) with 10% FBS under a 37° C., 5% CO2 atmosphere. MSCs collected and cell extractions were determined gene expression levels of cytokines and chondrogenic markers using a LightCycler® (Roche Applied Science). The fold change in gene expression on the y-axis was calculated based on untreated cell gene expression.

### 4. Statistical Analysis

Data were presented as mean±SEM and analyzed using GraphPad Prism software (Version 9.0). One-way ANOVA was calculated P values for multiple comparisons with the indicated post hoc Bonferroni test, utilizing at least three replicates. A non-paired Student's t-test was employed for two-group comparisons, while in vivo data were undergoing analysis using two-way ANOVA.

### 5. Results

### 5.1 CXCL14 Stimulated MSC Chondrogenesis and Cartilage Regeneration

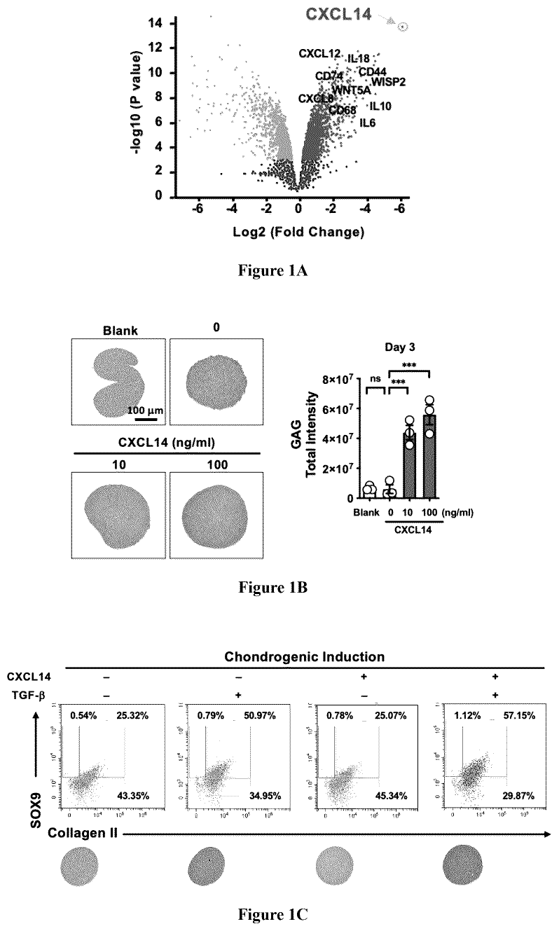

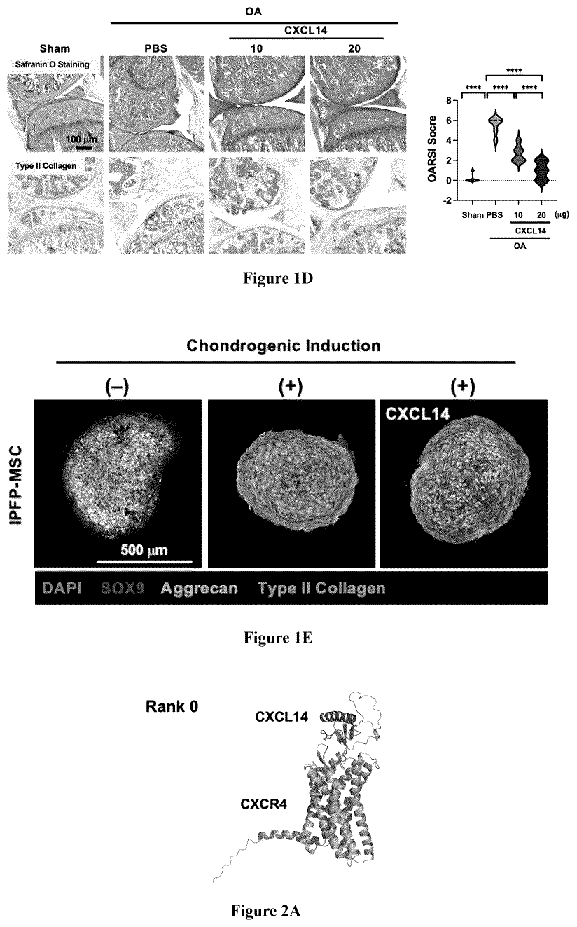

To dissect the kinetic gene expressions and explore the regulatory changes during chondrogenesis, we performed microarray database analysis from hBM-MSC. Microarray data showed the CXCL14 gene, which were highly expressed in hBM-MSC during chondrogenesis in the volcano plot (FIG. 1A). We next analyzed whether CXCL14 can induce chondrogenesis; we used IPFP-MSC from OA patients treated with recombinant protein CXCL14 and investigated the GAG content by Alcian blue staining. In our finding, we first determined the function of CXCL14 in regulating chondrogenic induction in IPFP-MSC from OA patients in vitro (FIG. 1B). Moreover, we explored that CXCL14 promoted the development of chondrogenic progenitors expressing SOX9 and Type II collagen, increasing GAG content (FIG. 1C). These observations suggested that augmentation of CXCL14 and TGF-β signaling may provide an explanation for the enhancement of chondrogenesis and cartilage regeneration. Then, we also characterized the function of CXCL14 in regulating chondrogenesis. Moreover, recombinant protein CXCL14 in a dose-dependent can stimulate cartilage repair in the anterior cruciate ligament transection-induced OA model (ACLT-induced OA) staining GAG content and type II collagen (FIG. 1D). According to the invention, it was revealed that CXCL14 stimulated the elevated levels of SOX9, type II collagen, and aggrecan in MSC spheroids during chondrogenesis (FIG. 1E). The results showed that CXCL14 could induce chondrogenesis, suggesting that CXCL14 might be able to undergo changes during the chondrogenic induction progress in cartilage. Collectively, we revealed a remarkable link between CXCL14 and chondrogenesis, which suggested the impact of CXCL14 on cartilage repair. In conclusion, the function of CXCL14 in regulating chondrogenesis was characterized.

5.2 the Functional Peptides Obtained from the CXCL14/CXCL12 Protein can Stimulate Chondrogenesis in Adipose-Derived Stem Cells (ADSCs).

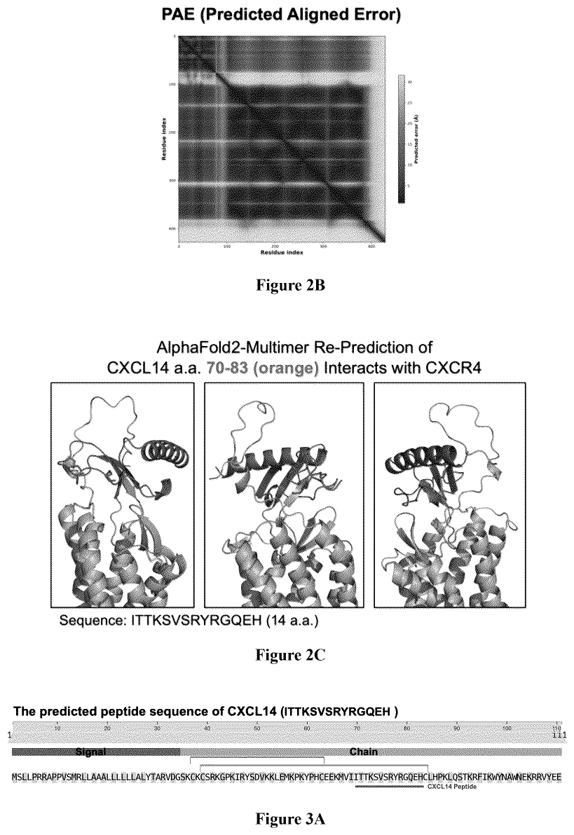

To effectively identify functional sequences within the CXCL14 protein, we employed AlphaFold2-Multimer prediction software. This allowed us to pinpoint functional peptide sequences in CXCL14 for enhancing chondrogenesis. Structural alignment between the CXCL14 and CXCR4 regions of the AlphaFold 2 model was presented (FIG. 2A). In addition, the Predicted Alignment Error (PAE) for the top-ranked model was illustrated, indicating the anticipated distance error in residue positions (FIG. 2B). Predicted amino acid sequencing was provided for the region between CXCL14 and CXCR4, comprising 14 amino acid sequence of ITTKSVSRYRGQEH (SEQ ID NO: 1) (FIG. 2C). Subsequently, we synthesized peptides based on the predicted sequence (ITTKSVSRYRGQEH) from CXCL14 and the published peptide having the amino acid sequence of RANVKHLKILN (SEQ ID NO: 2) from the CXCL12 protein to assess their chondrogenic capacity using qPCR

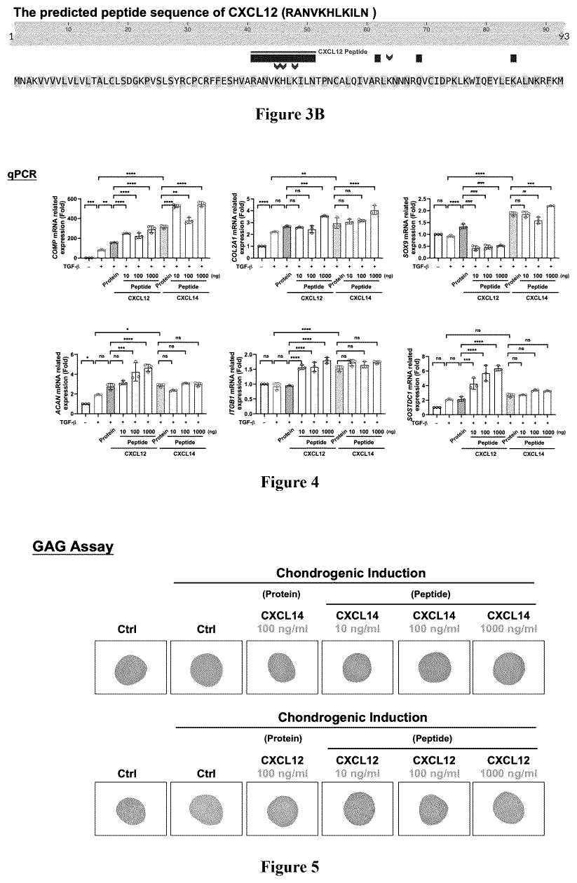

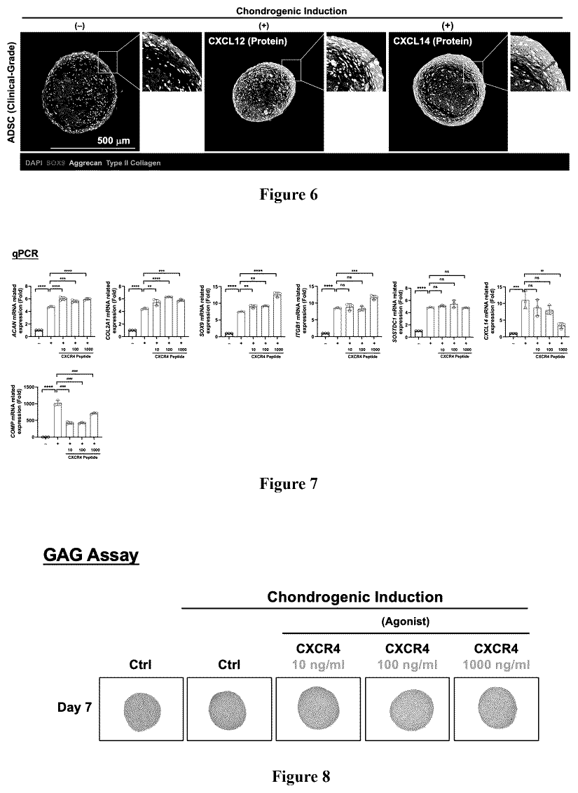

(FIG. 3) [13]. The results indicated that peptides derived from CXCL12/CXCL14 exhibited a greater ability to induce chondrogenic genes (FIG. 4) and GAG content (FIG. 5) compared to CXCL12/CXCL14 proteins in adipose-derived stem cells (ADSCs). Moreover, fluorescence confocal microscopy was employed to detect protein patterns and confirm the levels of SOX9, type II collagen, and aggrecan proteins in ADSC spheroids. CXCL14/CXCL12 proteins notably induced the expression of SOX9, type II collagen, and aggrecan, as depicted in merged images labeling SOX9 (red), type II collagen (green), and aggrecan (light blue) in ADSC spheroids (FIG. 6). Finally, to investigate whether the CXCR4 receptor of CXCL12/CXCL14 confers chondrogenic signaling during chondrogenesis, we treated ADSCs with a CXCR4 agonist for 7 days. FIG. 7 demonstrated that the CXCR4 agonist exhibited a superior capacity to induce chondrogenesis. In clinical-grade ADSCs, CXCR4 agonists significantly elevated the expression of chondrogenic genes and GAG content (FIG. 8).

### 5.3 ADSC Spheroid Formation Stimulated by CXCL14 and CXCL12 Enhances Cartilage Repair.

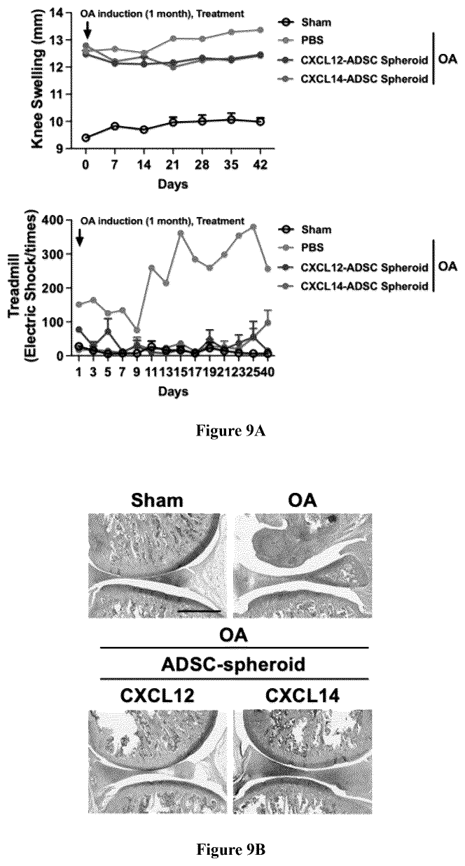

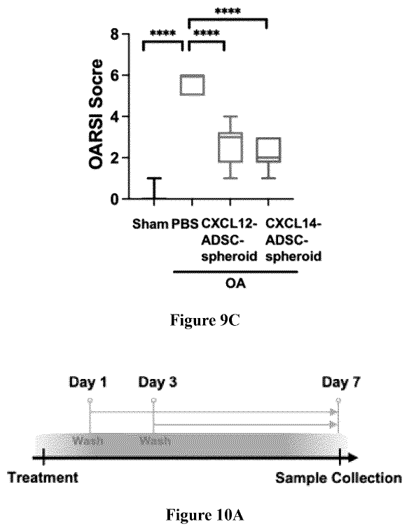

We investigated whether priming ADSC spheroids with CXCL14 and CXCL12, which closely mimic in vivo chondrogenic lineages, could enhance the regeneration of damaged cartilage. The results demonstrated that CXCL14/CXCL12-primed ADSC spheroids significantly promoted cartilage repair in an ACLT-induced OA model, as indicated by increased GAG content. In addition, knee swelling was alleviated following intra-articular administration of CXCL14/CXCL12-ADSC spheroids (FIG. 9A, upper panel). The treadmill grid delivered electric shocks of constant intensity during exercise, and the number of shocks experienced by the rats was significantly reduced in the group treated with CXCL14/CXCL12-ADSC spheroids compared to the OA group treated with PBS (FIG. 9A, lower panel). Safranin O staining of tibial articular cartilage revealed reduced cartilage thickness and staining intensity in OA rats. However, these parameters were improved in rats treated with CXCL14/CXCL12-ADSC spheroids (FIG. 9B). OARSI scores, reflecting the extent of articular cartilage damage and repair, were evaluated across all groups. The ACLT group exhibited a significantly higher OARSI score (5.61) compared to the sham group (0.17). Treatment with CXCL14/CXCL12-ADSC spheroids improved OARSI scores, reducing them to 2.06 and 2.56, respectively (FIG. 9C). The observed chondrogenic responses were associated with ECM remodeling induced by CXCL14/CXCL12-ADSC spheroids, which stimulated GAG expression. These findings highlight the reparative potential of CXCL14/CXCL12-ADSC spheroids in ACLT rats. Our results suggest that CXCL14 and CXCL12 play a key role in inducing chondrogenesis and may undergo dynamic changes during the chondrogenic induction process in cartilage. Collectively, this study uncovers a strong link between CXCL14/CXCL12 and chondrogenesis, emphasizing their therapeutic potential for cartilage repair. Furthermore, we elucidate the regulatory functions of CXCL14 and CXCL12 in chondrogenesis.

5.4 CXCL14 and CXCL12 Peptides and their Variants can Promote Chondrogenesis in MSCs Derived from Various Sources.

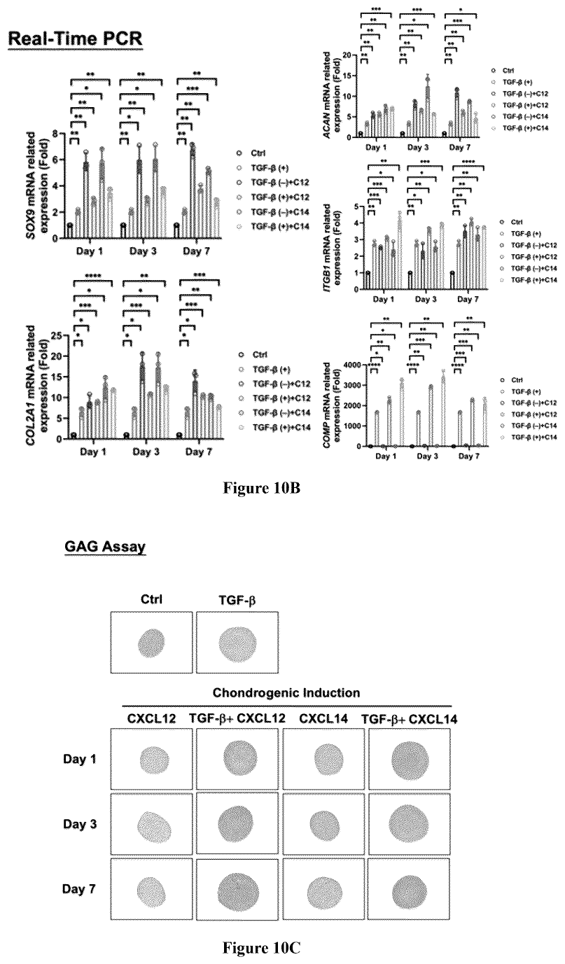

We further explored whether peptides derived from CXCL14 and CXCL12 have the capacity to induce chondrogenesis in MSCs from different sources. To assess their potential, ADSCs were utilized in the study to evaluate the chondrogenic capacity of CXCL14-P and CXCL12-P on day 7 (FIG. 10A). The results demonstrated that CXCL14-P and CXCL12-P significantly enhanced the expression of chondrogenic genes, ITGB1 and COMP, in the presence of TGF-β. Interestingly, the chondrogenic patterns, chondrogenic genes (FIG. 10B) and GAG content (FIG. 10C), induced by CXCL14-P or CXCL12-P were also evident even in the absence of TGF-β during chondrogenesis in ADSCs. FIG. 11 illustrates the cell-penetrating properties of CXCL14-P and CXCL12-P peptides in ADSCs, analyzed at different time points. ADSCs were incubated with FITC-conjugated peptides for one day, after which the chondrogenic medium was refreshed. Fluorescently labeled peptides were observed to penetrate ADSCs and remain detectable up to day 7. These findings suggest that CXCL14-P and CXCL12-P can promote chondrogenesis downstream of the TGF-β signaling pathway while also potentially enhancing chondrogenic gene expression through a TGF-β-independent mechanism. Collectively, these observations indicate that CXCL14-P and CXCL12-P have the ability to stimulate chondrogenesis and further amplify the effects of TGF-β during the chondrogenic process.

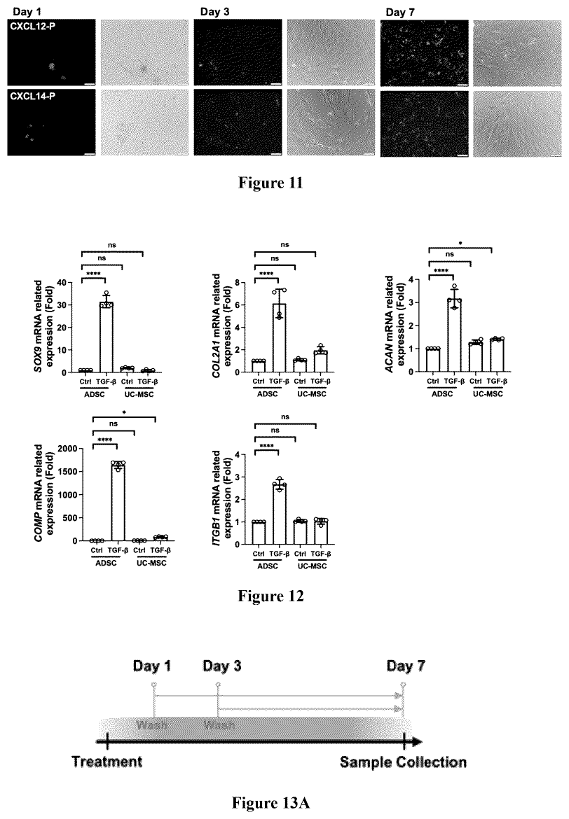

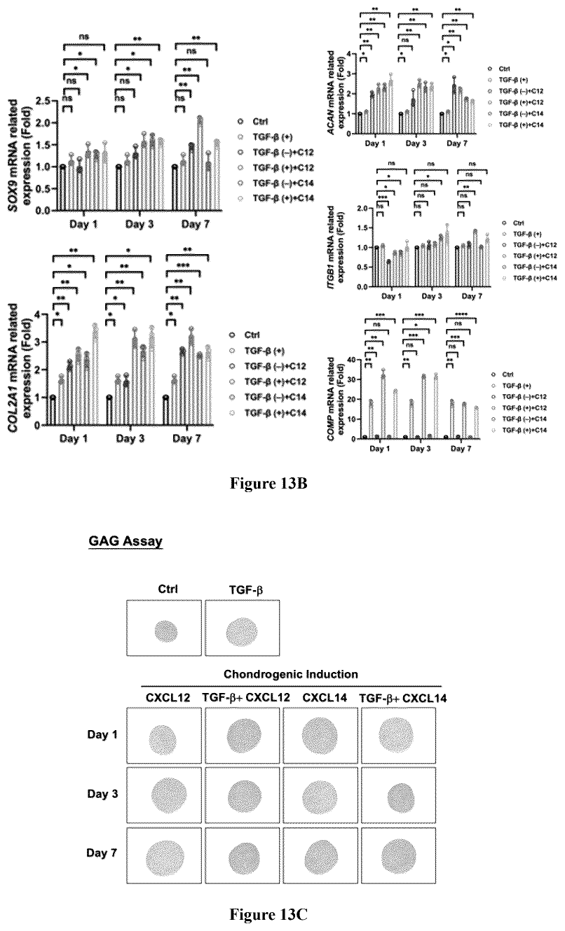

To further validate the role of CXCL14-P and CXCL12-P in regulating chondrogenesis, we compared their effects on another MSC source, umbilical cord-derived mesenchymal stem cells (UC-MSCs). It is important to note that the chondrogenic induction medium contained TGF-β. During chondrogenesis, we observed that the chondrogenic patterns in UC-MSCs were lower than those in ADSCs (FIG. 12). Subsequently, we assessed the chondrogenic potential of UC-MSCs following stimulation with CXCL14-P and CXCL12-P (FIG. 13A). Our results demonstrated that CXCL14-P and CXCL12-P effectively enhanced the chondrogenic potential of UC-MSCs. Although the expression of chondrogenic genes (FIG. 13B) and GAG content (FIG. 13C) in UC-MSCs was not as pronounced as in ADSCs, the findings confirm that CXCL14-P and CXCL12-P can robustly stimulate chondrogenesis across different MSC sources.

### 5.5 CXCL14 Peptide-Treated ADSC-Derived Extracellular Vesicles (EVs) Significantly Enhance Chondrogenesis.

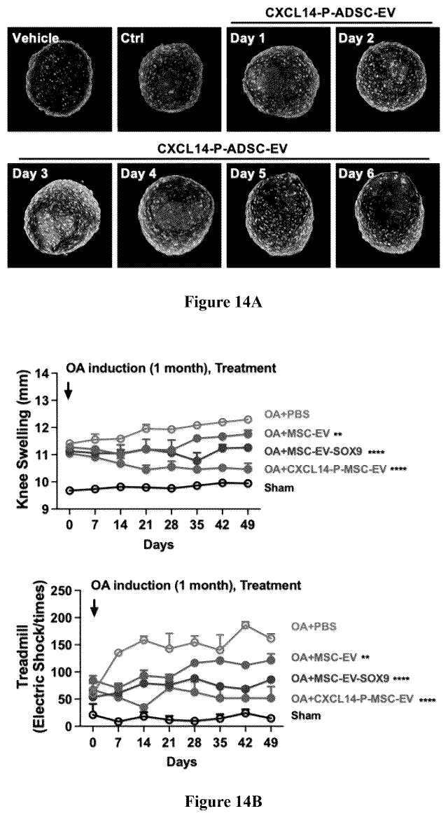

Extracellular vesicles (EVs) derived from MSCs hold significant potential for repairing damaged tissues. In this study, we explored whether EVs from ADSCs could effectively enhance chondrogenesis and promote cartilage repair. Immunofluorescence staining was used to assess protein levels of Collagen Type II (green), SOX9 (red), COMP (indigo), and Hoechst 33342 (gray) in 3D ADSC spheroids treated with MSC-EVs derived from CXCL14 peptide-primed ADSCs (CXCL14-P-MSC-EVs) at various time points. The results demonstrated that CXCL14-P-MSC-EVs effectively stimulated the production of chondrogenic proteins (FIG. 14A). To further explore the cartilage repair functions of EVs from CXCL14-P-treated ADSCs and the encapsulation of the transcription factor SOX9 within MSCs and purified EVs, we performed in vivo studies. In an ACLT-induced OA mouse model, intra-articular administration of MSC-EVs, MSC-EVs encapsulating SOX9 (MSC-EV-SOX9), or CXCL14-P-MSC-EVs significantly reduced knee swelling compared to the OA group treated with PBS. In addition, during treadmill exercise, the number of electrical shocks received (at a constant intensity) was significantly lower in the MSC-EV, MSC-EV-SOX9, and CXCL14-P-MSC-EV treatment groups, highlighting improved physical performance (FIG. 14B). These findings underscore the ability of CXCL14-P-MSC-EVs to significantly enhance chondrogenesis in human ADSCs, resulting in improved cartilage regeneration. Furthermore, treatment with CXCL14-P-MSC-EVs significantly upregulated the expression of critical chondrogenic proteins, likely through downstream signaling activation via CXCR4. This suggests that CXCL14-P-MSC-EVs possess the potential to serve as a novel therapeutic agent for osteoarthritis.

5.6 Alanine-Substituted Variants with Positively or Negatively Charged Amino Acids in the Sequences of CXCL14 and CXCR4 Peptides Enhance Chondrogenesis in Adipose Tissue-Derived MSCs.

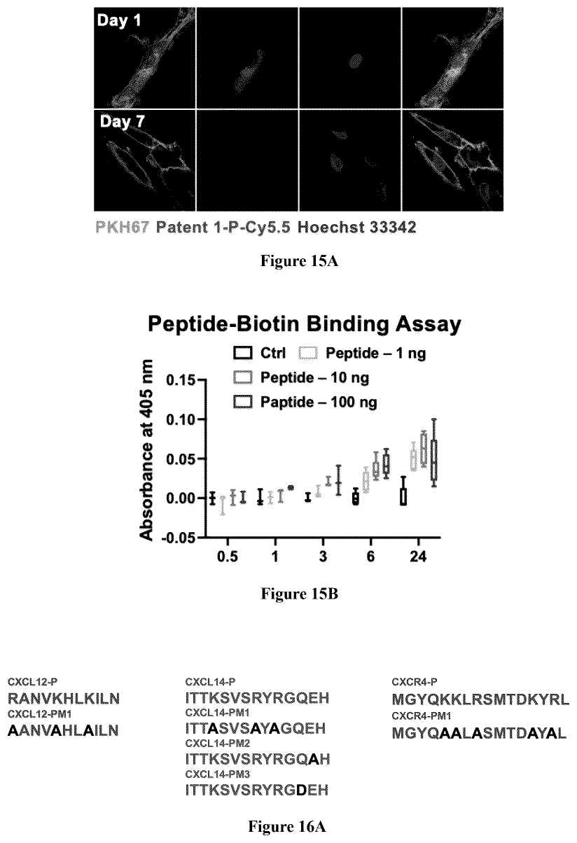

To investigate whether the chondrogenic induction in human adipose tissue-derived mesenchymal stromal cells (ADSCs) following CXCL14-P treatment is influenced by cellular uptake within a short period, ADSCs were exposed to CXCL14-P for one day. After 24 hours of treatment, the culture medium was replaced with fresh medium without CXCL14-P. Immunofluorescence staining revealed that CXCL14-P was detectable on day 1 but became undetectable by day 7 (FIG. 15A). To further confirm the binding of CXCL14-P to ADSCs, a biotin-conjugated CXCL14-P was used. ADSCs were treated with different concentrations of CXCL14-P-biotin at various time points. The results demonstrated a dose-and time-dependent increase in signal intensity, indicating that higher concentrations and prolonged exposure enhanced CXCL14-P binding to ADSCs (FIG. 15B).

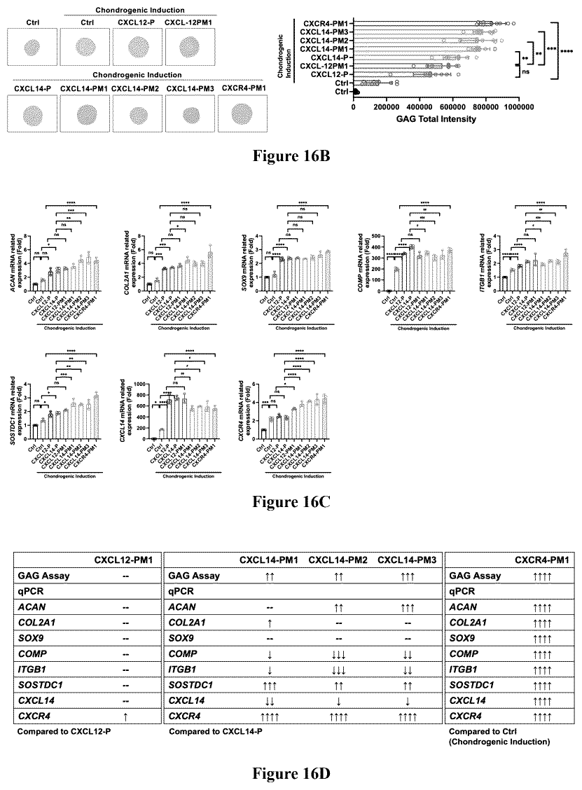

Building on the chondrogenic induction effects of CXCL12, CXCL14, and CXCR4 peptides in ADSCs, we introduced alanine substitutions to those positively or negatively charged amino acids on these peptides and investigated their potential on chondrogenesis in ADSCs. The sequences of the alanine-substituted variants of CXCL12, CXCL14, and CXCR4 peptides are presented in FIG. 16A. To determine whether alanine-substituted variants exhibited alteration on chondrogenic potential than their unmodified counterparts, ADSCs were treated with either the original CXCL12, CXCL14, and CXCR4 peptides (e.g. referred to as CXCL12-P) or their alanine-substituted variants (e.g. referred to as CXCL12-PM1). First, the chondrogenic differentiation of ADSC-containing pellets was assessed using alcian blue staining. On day 7 of chondrogenesis, treatment with alanine-substituted variants of CXCL14 and CXCR4, but not CXCL12, peptides resulted in increased glycosaminoglycan (GAG) deposition in ADSCs (FIG. 16B). Second, the expression levels of SOX9, COL2A1, COMP, ACAN, ITGB1, CXCL14, and CXCR4 genes were analyzed by using real-time PCR on day 7. The results demonstrated that alanine-substituted variants of CXCL14 and CXCR4 peptides significantly enhanced chondrogenesis in ADSCs, as evidenced by increased, at least in part, expression of chondrogenic marker genes (FIG. 16C), further supporting their role in promoting chondrogenesis. Consistent with the GAG assay, there is no significant changes on CXCL12-P vs CXCL12-PM1 on the expression of chondrogenic marker genes, suggesting that these positively charged amino acids are not functional determinants in this CXCL12-P peptide. However, the presence of positively or negatively charged amino acids can act as negative determinants in the case of CXCL14-P compared to its modified forms, CXCL14-PM1 and CXCL14-PM2. The substitution of these charged amino acids has been shown to enhance GAG contents. Interestingly, replacing Glutamine (Q) with the negatively charged Aspartic acid (D) in CXCL14-P did not significantly alter GAG contents, suggesting that Aspartic acid may not be a critical determinant in CXCL14-PM3.

Similarly, replacing positively charged amino acids in CXCR4-P with Alanine (referred to as CXCR4-PM1) significantly enhanced GAG activity, further supporting the idea that these positively charged amino acids act as negative determinants in CXCR4-P. FIG. 16D summarizes gene expression and GAG content in ADSCs during chondrogenesis induced by alanine-substituted variants, where positively or negatively charged amino acids were modified in CXCL12, CXCL14, and CXCR4 peptide sequences. These newly synthesized variants (FIG. 16A) served as novel chondrogenesis inducers for adipose tissue-derived MSCs.

In conclusion, the genetically modified recombinant variants of CXCL14 or CXCL12 provided the same function.

### 5.7 Modification of CXCL14-P to Enhance Stability and Chondrogenic Potential.

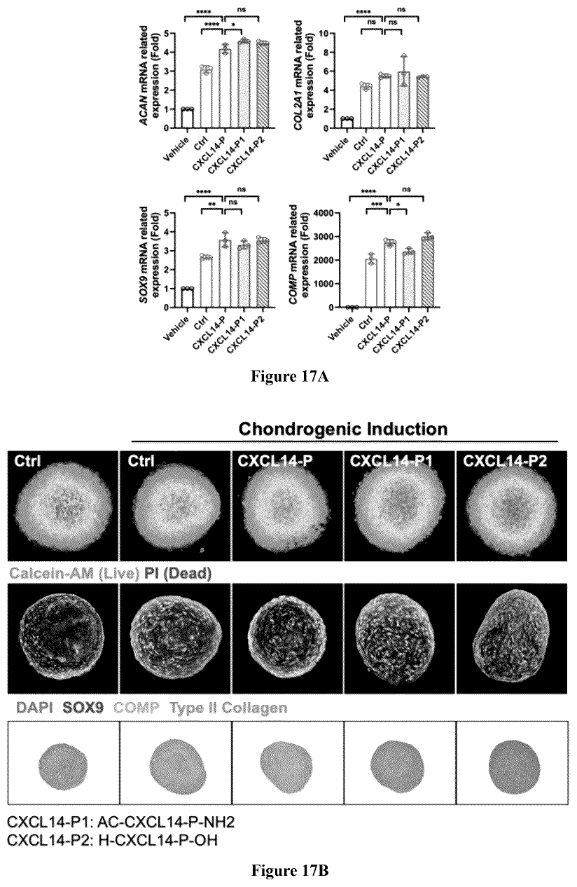

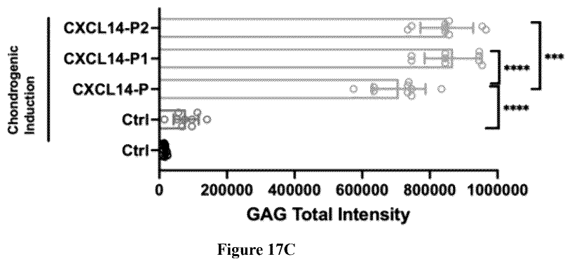

To reduce the degradation of CXCL14-P, we designed and synthesized modified CXCL14 peptides, including CXCL14-P1 (AC-CXCL14-P-NH2) and CXCL14-P2 (H-CXCL14-P-OH). These modifications were not found to significantly enhance chondrogenic gene expression. ADSCs were treated with CXCL14-P, CXCL14-P1, or CXCL14-P2 for 24 hours, followed by a medium change to peptide-free conditions. On day 7, ADSCs treated with CXCL14-P1 or CXCL14-P2 showed no significant differences in the expression of key chondrogenic markers, including ACAN, COL2A1, SOX9, and COMP, compared to CXCL14-P (FIG. 17A).

To assess whether these peptide modifications affected cell viability, ADSC spheroids were stained with calcein-AM (live) and PI (dead) dyes. As shown in FIG. 17B (top), spheroid formation during chondrogenesis with peptide treatment did not induce cell death. Furthermore, immunofluorescence staining revealed an increase in Collagen Type II, SOX9, and COMP expression in 3D ADSC spheroids following treatment with CXCL14-P, CXCL14-P1, or CXCL14-P2 by day 7 (FIG. 17B, middle). Notably, alcian blue staining demonstrated that CXCL14-P1 and CXCL14-P2 significantly enhanced GAG deposition in ADSCs compared to CXCL14-P by day 7, indicating improved chondrogenic differentiation. In addition, CXCL14-P1 and CXCL14-P2 markedly increased the chondrogenic potential of ADSCs, as evidenced by the higher GAG accumulation observed in ADSC pellets on day 7 (FIG. 17B, bottom). Taken together, modification of the original peptide via amino acid substitution or N- and C-terminus modification could even enhance chondrogenesis. In conclusion, the chemically modified proteins of CXCL14 or CXCL12 provided the same function.

While this invention has been described in connection with what is presently considered to be practical exemplary embodiments, it is to be understood that the invention is not limited to the disclosed embodiments. On the contrary, it is intended to cover various modifications and equivalent arrangements included within the spirit and scope of the appended claims.

### REFERENCES

ADDIN E N.REFLIST 1. Munsell E V, Kurpad D S, Freeman T A, Sullivan M O. Histone-targeted gene transfer of bone morphogenetic protein-2 enhances mesenchymal stem cell chondrogenic differentiation. Acta Biomater 2018; 71:156-167.
2. Zhang T, Wen F, Wu Y et al. Cross-talk between TGF-beta/SMAD and integrin signaling pathways in regulating hypertrophy of mesenchymal stem cell chondrogenesis under deferral dynamic compression. Biomaterials 2015; 38:72-85.
3. Luo Z, Jiang L, Xu Y et al. Mechano growth factor (MGF) and transforming growth factor (TGF)-beta3 functionalized silk scaffolds enhance articular hyaline cartilage regeneration in rabbit model. Biomaterials 2015; 52:463-475.
4. van Beuningen H M, Glansbeek H L, van der Kraan P M, van den Berg W B. Differential effects of local application of BMP-2 or TGF-beta 1 on both articular cartilage composition and osteophyte formation. Osteoarthritis Cartilage 1998; 6:306-317.
5. Deshmukh V, O'Green A L, Bossard C et al. Modulation of the Wnt pathway through inhibition of CLK2 and DYRKIA by lorecivivint as a novel, potentially disease-modifying approach for knee osteoarthritis treatment. Osteoarthritis Cartilage 2019; 27:1347-1360.
6. Deshmukh V, Hu H, Barroga C et al. A small-molecule inhibitor of the Wnt pathway (SM04690) as a potential disease modifying agent for the treatment of osteoarthritis of the knee. Osteoarthritis Cartilage 2018; 26:18-27.
7. Gelse K, Ekici A B, Cipa F et al. Molecular differentiation between osteophytic and articular cartilage—clues for a transient and permanent chondrocyte phenotype. Osteoarthritis Cartilage 2012; 20:162-171.
8. Somoza R A, Correa D, Labat I et al. Transcriptome-Wide Analyses of Human Neonatal Articular Cartilage and Human Mesenchymal Stem Cell-Derived Cartilage Provide a New Molecular Target for Evaluating Engineered Cartilage. Tissue Eng Part A 2018; 24:335-350.
9. Dai C, Basilico P, Cremona T P et al. CXCL14 displays antimicrobial activity against respiratory tract bacteria and contributes to clearance of Streptococcus pneumoniae pulmonary infection. J Immunol 2015; 194:5980-5989.
10. Maerki C, Meuter S, Liebi M et al. Potent and broad-spectrum antimicrobial activity of CXCL14 suggests an immediate role in skin infections. J Immunol 2009; 182:507-514.
11. Tanegashima K, Suzuki K, Nakayama Y et al. CXCL14 is a natural inhibitor of the CXCL12-CXCR4 signaling axis. FEBS Lett 2013; 587:1731-1735.
12. Kufareva I, Stephens B S, Holden L G et al. Stoichiometry and geometry of the CXC chemokine receptor 4 complex with CXC ligand 12: molecular modeling and experimental validation. Proc Natl Acad Sci USA 2014; 111: E5363-5372.
13. Fujita M, Davari P, Takada Y K, Takada Y. Stromal cell-derived factor-1 (CXCL12) activates integrins by direct binding to an allosteric ligand-binding site (site 2) of integrins without CXCR4. Biochem J 2018; 475:723-732.

1. A method for treating a cartilage defect disease through cartilage regeneration in a subject, comprising administering said subject a therapeutically effective amount of a composition,
wherein the composition comprises a CXCR4 agonist, and
wherein the agonist has a CXCR4 agonistic activity that induces chondrogenesis.

2. The method of claim 1, wherein the agonist is CXCL14 protein, or a variant, variable domain, functional derivative, or fragment thereof having the same function of CXCL14, a genetically modified recombinant protein or chemically modified protein thereof, or a gene construct for expressions of CXCL14.

3. The method of claim 1, wherein the agonist is CXCL12 protein, or a variant, variable domain, functional derivative, or fragment thereof having the same function of CXCL12, a genetically modified recombinant protein or chemically modified protein, or a gene construct for expressions of CXCL 12.

4-7. (canceled)

8. A method for treating a cartilage defect disease in a subject, comprising administering said subject with a cell preparation,
wherein the cell preparation is obtained by culturing cells treated with a CXCR4 agonist, and
wherein the agonist has a CXCR4 agonistic activity that induce chondrogenesis.

9. The method of claim 8, wherein the agonist is CXCL14 protein, or a variant, variable domain, functional derivative, or fragment thereof having the same function of CXCL14, a genetically modified recombinant protein or chemically modified protein thereof, or a gene construct for expressions of CXCL14.

10. The method of claim 8, wherein the agonist is CXCL 12 protein, or a variant, variable domain, functional derivative, or fragment thereof having the same function of CXCL12, a genetically modified recombinant protein or chemically modified protein thereof, or a gene construct for expressions of CXCL12.

11. The method of claim 8, wherein the cartilage defect disease is degenerative osteoarthritis.

12. The method of claim 8, wherein the cells are stem cells, including induced pluripotent stem cells.

13. The method of claim 12, wherein the stem cells are mesenchymal stem cells (MSCs).

14. The method of claim 13, wherein the MSCs are selected from the group consisting of Wharton's jelly-derived MSCs (WJ-MSCs), infrapatellar fat pad-derived MSCs (IPFP-MSCs), subcutaneous adipose tissue-derived MSCs (SC-MSCs), amniotic fluid-derived MSCs (AF-MSCs), bone marrow-derived MSCs (BM-MSCs), and umbilical cord-derived MSCs (UC-MSCs).

15. The method of claim 8,
wherein the cell preparation is the cell medium obtained by culturing cells treated with a CXCR4 agonist, and
wherein the agonist has a CXCR4 agonistic activity that induce chondrogenesis.

16. The method of claim 15, wherein the agonist is CXCL 14 protein, or a variant, variable domain, functional derivative, or fragment thereof having the same function of CXCL14, a genetically modified recombinant protein or chemically modified protein thereof, or a gene construct for expressions of CXCL14.

17. The method of claim 15, wherein the agonist is CXCL 12 protein, or a variant, variable domain, functional derivative, or fragment thereof having the same function of CXCL12, a genetically modified recombinant protein or chemically modified protein thereof, or a gene construct for expressions of CXCL12.

18. The method of claim 15, wherein the cartilage defect disease is degenerative osteoarthritis.

19. The method of claim 15, wherein the cells are stem cells, including induced pluripotent stem cells.

20. The method of claim 19, wherein the stem cells are mesenchymal stem cells (MSCs).

21. The method of claim 20, wherein the MSCs are selected from the group consisting of Wharton's jelly-derived MSCs (WJ-MSCs), infrapatellar fat pad-derived MSCs (IPFP-MSCs), subcutaneous adipose tissue-derived MSCs (SC-MSCs), amniotic fluid-derived MSCs (AF-MSCs), bone marrow-derived MSCs (BM-MSCs), and umbilical cord-derived MSCs (UC-MSCs).

22. The method of claim 15, wherein the cell medium contains secretome derived from the treated cells.

23. The method of claim 15, wherein the cell preparation further comprises extracellular vesicles,
wherein the extracellular vesicles are released from the cells treated with a CXCR4 agonist.

24-28. (canceled)

29. The method of claim 15, wherein the cells are MSCs selected from the group consisting of Wharton's jelly-derived MSCs (WJ-MSCs), infrapatellar fat pad-derived MSCs (IPFP-MSCs), subcutaneous adipose tissue-derived MSCs (SC-MSCs), amniotic fluid-derived MSCs (AF-MSCs), bone marrow-derived MSCs (BM-MSCs), and umbilical cord-derived MSCs (UC-MSCs).

30-34. (canceled)


SEQUENCE LISTING (8 sequences)

SEQ ID NO: 1 - protein, 14 aa, synthetic construct
ITTKSVSRYRGQEH

SEQ ID NO: 2 - protein, 11 aa, synthetic construct
RANVKHLKILN

SEQ ID NO: 3 - protein, 16 aa, synthetic construct
MGYQKKLRSMTDKYRL

SEQ ID NO: 4 - protein, 14 aa, synthetic construct
ITTASVSAYAGQEH

SEQ ID NO: 5 - protein, 14 aa, synthetic construct
ITTKSVSRYRGQAH

SEQ ID NO: 6 - protein, 14 aa, synthetic construct
ITTKSVSRYRGDEH

SEQ ID NO: 7 - protein, 11 aa, synthetic construct
AANVAHLAILN

SEQ ID NO: 8 - protein, 16 aa, synthetic construct
MGYQAALASMTDAYAL